(19) United States
(12) Patent Application Publication
(10) Pub. No.: US 2017/0136211 A1
(43) Pub. Date: May 18, 2017

(54) CATHETER STIFFNESS ADJUSTMENT SYSTEM AND METHOD

(71) Applicant: DEPUY SYNTHES PRODUCTS, INC., RAYNHAM, MA (US)

(72) Inventor: EDWARD ARGUELLO, MIRAMAR, FL (US)

(21) Appl. No.: 15/416,928

(22) Filed: Jan. 26, 2017

Related U.S. Application Data
(62) Division of application No. 13/799,821, filed on Mar. 13, 2013.

(51) Int. Cl. A61M 25/00

(52) U.S. Cl. CPC A61M 25/0053; A61M 2025/0004; A61M 2025/0063

(57) ABSTRACT

A catheter stiffener adjustment system including a first member with an outer diameter and having at least three first segments with at least one joint enabling the segments to bend relative to each other, and a second member having an inner diameter defining a passageway through which the first member is movable relative to the second member. The second member has at least three second segments with at least one joint enabling the segments to bend relative to each other. In a first position, the first segments and the second segments are alignable substantially in phase to generate a first flexure condition. In a second position, the first segments and the second segments are alignable substantially out of phase to generate a second, stiffer flexure condition.

### BACKGROUND OF THE INVENTION

1. Field of the Invention

The invention relates to catheters insertable within vasculature of a patient and more particularly to mechanisms and systems that alter the stiffness of at least one section of the catheter.

2. Description of the Related Art

Traditional vascular catheter designs struggle with the trade-off between flexibility and stiffness. Flexibility is desired to assist in navigating tortuous anatomy of the vasculature. On the other hand, stiffness is needed both for pushability during insertion and, after the distal tip of the catheter is positioned at a selected site, for stability to provide support during the advancement of accessory devices through the catheter.

There are a number of systems for steering catheters, such as disclosed by Lundquist et al. in U.S. Pat. No. 5,254,088 and by Heinzelman et al. in U.S. Pat. No. 5,364,351. Steering mechanisms described in these patents have levers or knobs which cause selective rotation or tension on steering wires or other elements within the catheters.

Several constructions of an adjustable stiffness catheter are described by Gregorich et al. in U.S. Patent Publication No. 2007/0060880. In one construction having coaxial hypotubes, the inner diameter of an outer hypotube is decreased, or the outer diameter of an inner hypotube is increased, to engage one hypotube with the other hypotube. In other constructions, one or more inflatable elements engage a hypotube.

It is therefore desirable to have an improved catheter stiffness adjustment mechanism that is simple to use and which does not bind inner and outer elements together.

### SUMMARY OF THE INVENTION

An object of the present invention is to provide an improved stiffness adjustment system that can be actuated as desired during insertion and placement of a catheter to change its flexibility.

This invention features a catheter stiffener adjustment system including a first member with an outer diameter and having at least three first segments with at least one joint enabling the segments to bend relative to each other, and a second member having an inner diameter defining a passageway through which the first member is movable relative to the second member. The second member has at least three second segments with at least one joint enabling the segments to bend relative to each other. In a first position, the first segments and the second segments are alignable substantially in phase to generate a first flexure condition. In a second position, the first segments and the second segments are alignable substantially out of phase to generate a second, stiffer flexure condition.

In certain embodiments, the first member and the second member are at least one of rotatable and translatable relative to each other in both the first and second positions. In one embodiment, the joint for at least one of the first and second members is substantially helical. In some embodiments, the joint for at least one of the first and second members is formed by a thinning of material separating the segments. In other embodiments, the joint for at least one of the first and second members is formed by an elimination of material separating the segments along at least one side of the member.

In some embodiments, at least one of the first and second members is substantially tubular. In one embodiment, each of the first and second members is substantially cylindrical. In certain embodiments, the inner diameter of the second member is greater than the outer diameter of the first member in both the first and second positions.

This invention also features a catheter having a stiffener adjustment system including an outer catheter body defining a central lumen, a first member with an outer diameter and having at least three first segments with at least one joint enabling the segments to bend relative to each other, and a second member movable through the central lumen of the catheter body and having an inner diameter defining a passageway through which the first member is movable relative to the second member. The second member has at least three second segments with at least one joint enabling the segments to bend relative to each other. In a first position, the first segments and the second segments are alignable substantially in phase to generate a first flexure condition for the catheter. In a second position, the first segments and the second segments are alignable substantially out of phase to generate a second, stiffer flexure condition for the catheter.

This invention may also be expressed as a method of adjusting the stiffness of a catheter, including selecting a catheter having an outer catheter body defining a central lumen, a first member with an outer diameter and having at least three first segments with at least one joint enabling the segments to bend relative to each other, and a second member movable through the central lumen of the catheter body and having an inner diameter defining a passageway through which the first member is movable relative to the second member. The second member has at least three second segments with at least one joint enabling the segments to bend relative to each other. At least the three first segments and the three second segments have sufficiently similar lengths to enable alignment substantially in phase with the first and second members in a first position and, in a second position, being alignable substantially out of phase. The method further includes aligning the first segments and the second segments substantially in phase to generate a first flexure condition for the catheter. The method also includes aligning the first segments and the second segments substantially out of phase to generate a second, stiffer flexure condition for the catheter.

### BRIEF DESCRIPTION OF THE DRAWINGS

In what follows, preferred embodiments of the invention are explained in more detail with reference to the drawings, in which:

FIG. 1 is a schematic cross-sectional view through a region of a catheter according to the present invention having an outer catheter body containing first and second members movable relative to each other;

FIG. 2 is a diagrammatic side view of first and second members aligned in phase to generate a first flexure condition;

FIG. 3 is a diagrammatic side view of first and second members aligned out of phase to generate a second, stiffer flexure condition;

FIG. 4 is a schematic side view of first and second members separated from each other for illustrative purposes and aligned in phase to generate a first flexure condition when positioned concentrically;

FIG. 5 is a schematic similar to FIG. 4 with the first and second members aligned out of phase to generate a second, stiffer flexure condition when concentric;

FIG. 6 is a schematic perspective view of a proximal portion of the catheter of FIG. 1 having an actuation mechanism to move the first and second members relative to each other according to the present invention; and

FIG. 7 is a schematic side view of a distal portion of first and second actuation members according to the present invention.

### DETAILED DESCRIPTION OF THE PRESENTLY PREFERRED EMBODIMENTS

This invention may be accomplished by a vascular catheter having adjustable stiffness during insertion into vasculature of a patient, where the terms “vascular” and “vasculature” are utilized in their broadest meaning to include any duct or tube network in a human or other animal. A catheter according to the present invention includes a catheter stiffener adjustment system having a first member with an outer diameter and having at least three first segments with at least one joint enabling the segments to bend relative to each other, and a second member having an inner diameter defining a passageway through which the first member is movable relative to the second member. The second member has at least three second segments with at least one joint enabling the segments to bend relative to each other. In a first position, the first segments and the second segments are alignable substantially in phase to generate a first flexure condition, also referred to as a “flexibility mode”. In a second position, the first segments and the second segments are alignable substantially out of phase to generate a second, stiffer flexure condition, also referred to as a “stiffness mode”.

The present invention solves the trade-off faced by most conventional catheters by providing a system that generates an amount of stiffness that can be changed as desired, prior to or during a surgical procedure, by utilizing a simple actuation mechanism such as described below in relation to FIG. 6. Therefore, a surgeon or other clinician can activate the stiffness mode for pushability, including accepting a catheter already placed in stiffness mode by a manufacturer, advance the catheter into vasculature utilizing the stiffness mode, and then transition to the flexible mode to navigate tortuous distal anatomy. The clinician can finally return to the stiffness mode once the working portion of the catheter, typically at or near the distal end of the catheter, has reached the target anatomy selected by the clinician to provide stability and support during the delivery of accessory devices. In addition, the adjustable stiffness system is segmentable for different lengths, allowing for particular sections or regions of a catheter to alternate between flexibility and stiffness modes independently.

A catheter 10, shown in cross-section in FIG. 1 as one construction according to the present invention, includes an outer catheter body 12, typically formed of a biocompatible polymeric material, having an outer surface 11 and an inner surface 13 defining a central lumen 20. A second, outer member 14 has an outer surface 15 and an inner surface 17 defining a passageway 22. A first, inner member 16 is movably disposed within passageway 22 and has an outer surface 19 and an inner surface 21 defining a passageway 24. The first and second members 14, 16, formed of metal or polymeric hypotubes in some constructions, are movable relative to each other, such as by rotation indicated by arrow 18.

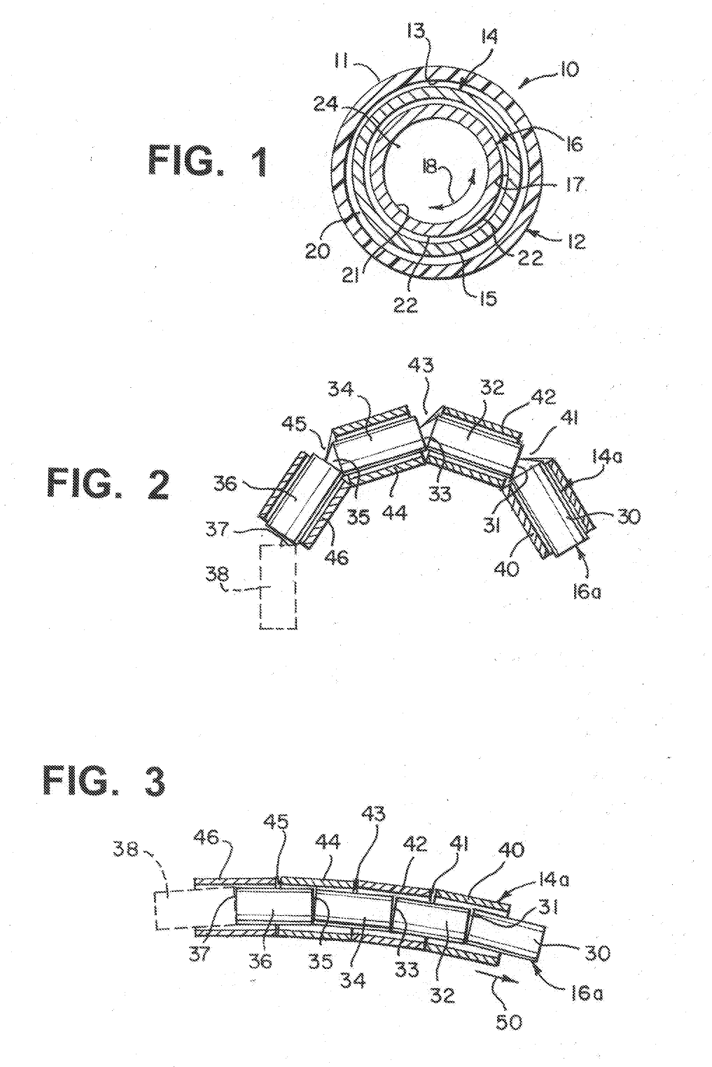

FIG. 2 is a diagrammatic side view of first and second members 16a, 14a aligned in phase to generate a first flexure condition, also referred to as a flexibility mode. In this construction, first member 16a includes segments 30, 32, 34 and 36 separated by joints 31, 33 and 35. Dashed line 38 represents additional segments in some constructions, with at least one further joint 37, and represents non-segmented regions in other constructions. Similarly, second member 14a has segments 40, 42, 44 and 46 separated by joints 41, 43 and 45 spaced from each other at substantially the same frequency or periodicity as for joints 31, 33 and 35 of the first member 16a.

FIG. 3 is a diagrammatic side view of first and second members 16a, 14a aligned out of phase to generate a second, stiffer flexure condition also referred to as a stiffness mode. In this construction, first member 16a has been retracted in a proximal direction, toward a clinician operating the alignment system, as indicated by arrow 50.

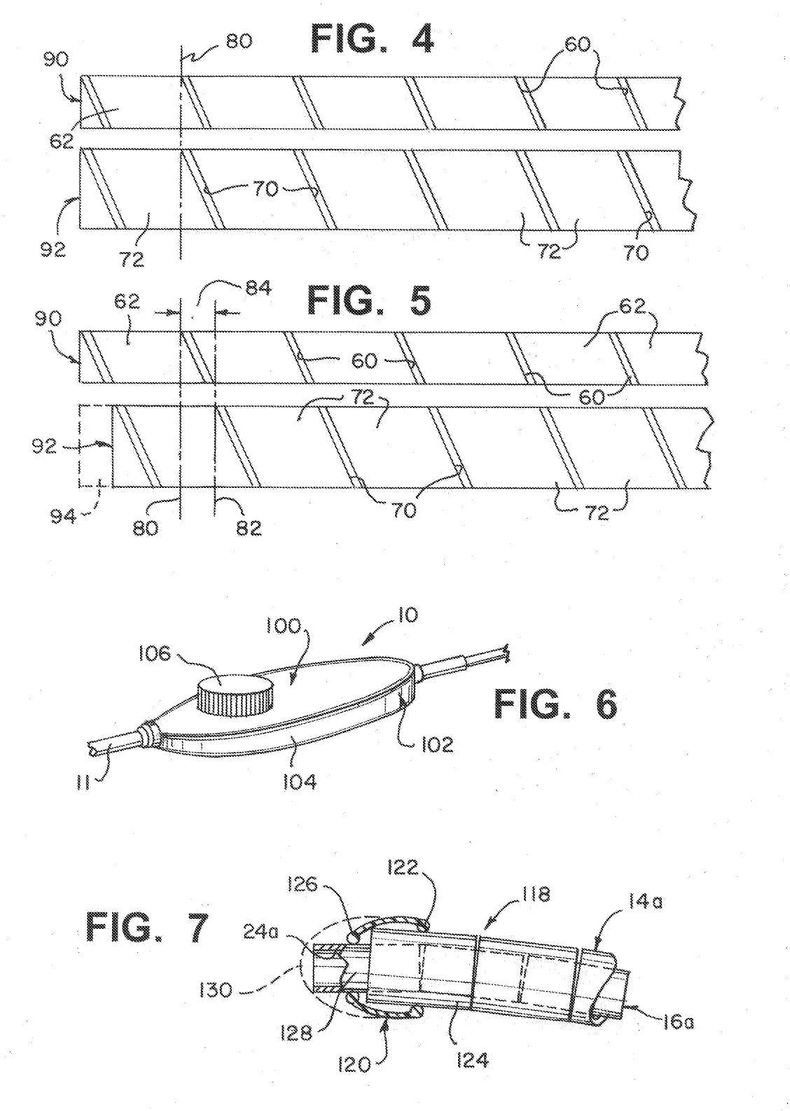

FIG. 4 is a schematic side view of first and second members 14b, 16b separated from each other for illustrative purposes and aligned in phase to generate a first flexure condition when positioned concentrically as shown in FIG. 1. In this construction, a single helical joint 60 separates segments 62 of first member 16b while single helical joint 70 separates segments 72 of second member 14b. Joints 60 and 70 are aligned in phase as indicated by dashed line 80.

FIG. 5 is a schematic similar to FIG. 4 with the first and second members 16b, 14b aligned out of phase to generate a second, stiffer flexure condition when concentric. In this construction, second member 14b has been retracted proximally, arrow 78, to move joint 70 by a distance 84 to a new position represented by dashed line 82. Distance 84 is less than the spacing between segments as indicated by arrow 86. Solid ends 90 and 92 represent relative positions when first and second members 16a, 14a are translated relative to each other; dashed line 94 represents the unchanged longitudinal position of second member 14b if the first and second members are simply rotated relative to each other to achieve the out-of-phase alignment shown in FIG. 5.

In other words, this invention may be accomplished by a two-member system, each member having one or more joints of substantially the same frequency or periodicity as the other member to thereby establish segments having similar lengths, preferably substantially identical lengths, and enable the segments of the first and second members to be controllably aligned in phase or out of phase, depending on the desired amount of flexibility or stiffness for the members and accompanying structure such as an outer catheter body. In some constructions, the joints are formed by cuts, either continuous or discrete, which extend through the entire wall thickness of the member and, in other constructions, permeate to a selected wall thickness to create living hinges. One suitable source for metal hypotubes, formed of a platinum alloy or tantalum if radiopacity is desired, is Johnson Matthey Medical Components (see “www.jmmedical.com”). If metal or polymeric hypotubes or other hollow, substantially cylindrical tubes are utilized, it is generally easier to manufacture the joints as cuts made by laser or water jet into the outer diameters of the tubes. The segments then have a major or outer diameter, with a maximum wall thickness, while the joints have a minor or smaller diameter with minimal or no wall thickness, depending on the depth of the cuts. Partial cuts with minimal wall thickness can also be referred to as channels or grooves between segments.

Additionally, the joints, as continuous or discrete bending loci, can be formed on all of the length of the tubes or other elongated items serving as first and second members, or can be formed only on certain sections of the first and second members. Further, each member may also vary in thickness, material and/or durometer to generate a desired amount of flexibility or stiffness over selected lengths for the respective modes to tailor the stiffener adjustment system to particular procedures. Moreover, the periodic cut frequency, cut type and shape, and material of the two members can be adjusted to manage catheter kink radius, mean and differential stiffness between the two modes or states of flexibility, and length change needed to transition between the two modes or states.

FIG. 6 is a schematic perspective view of a proximal portion 100 of catheter 10, FIG. 1, having an actuation mechanism 102 with a housing 104 and control knob 106 to move the first and second members relative to each other, such as by a sliding or twisting motion to translate and/or rotate one or both members, according to the present invention. In other constructions, a control lever or toggle is provided, which is purely mechanical in some forms and electro-mechanical in other forms.

FIG. 7 is a schematic side view of a distal portion 118 of first and second actuation members 16a, 14a of FIG. 2 with an elastomeric cover 120 that enables relative motion between the first and second members. In this construction, cover 120 has a first O-ring-type seal 122 contacting outer segment 124 of second member 14a and has a second O-ring-type seal 126 contacting inner segment 128 of first member 16a. Accessory devices can be advanced distally through a passageway 24a in first member 16a.

Dashed line 130 represents another construction in which cover 120 has a dome-like distal end which occludes the distal opening of passageway 24a. Cover 120 is sealed entirely by seal 122 on outer segment 124, obviating the need for seal 126.

Biocompatible jacketing materials, inner liners, outer liners, and/or coatings can be applied to or over inner and/or outer surfaces of one or both of the first and second members as desired for particular procedures and to enhance compatibility with devices or fluids passed through catheters with adjustable stiffness according to the present invention. For example, imaging agents, pharmaceuticals, saline or other liquids may be injected under pressure. Liners or spacers can assist alignment of the joints of both members during use, and preferably reduce frictional engagement between the members within tortuous vascular pathways.

This invention may also be accomplished by a method of adjusting the stiffness of a catheter, including selecting a catheter having an outer catheter body defining a central lumen, a first member with an outer diameter and having at least three first segments with at least one joint enabling the segments to bend relative to each other, and a second member movable through the central lumen of the catheter body and having an inner diameter defining a passageway through which the first member is movable relative to the second member. The second member has at least three second segments with at least one joint enabling the segments to bend relative to each other. At least the three first segments and the three second segments have sufficiently similar lengths to enable alignment substantially in phase with the first and second members in a first position and, in a second position, being alignable substantially out of phase. The method further includes aligning the first segments and the second segments substantially in phase to generate a first flexure condition for the catheter. The method also includes aligning the first segments and the second segments substantially out of phase to generate a second, stiffer flexure condition for the catheter.

Thus, while there have been shown, described, and pointed out fundamental novel features of the invention as applied to a preferred embodiment thereof, it will be understood that various omissions, substitutions, and changes in the form and details of the devices illustrated, and in their operation, may be made by those skilled in the art without departing from the spirit and scope of the invention. For example, it is expressly intended that all combinations of those elements and/or steps that perform substantially the same function, in substantially the same way, to achieve the same results be within the scope of the invention. Substitutions of elements from one described embodiment to another are also fully intended and contemplated. It is also to be understood that the drawings are not necessarily drawn to scale, but that they are merely conceptual in nature. It is the intention, therefore, to be limited only as indicated by the scope of the claims appended hereto.

Every issued patent, pending patent application, publication, journal article, book or any other reference cited herein is each incorporated by reference in their entirety.

1. A catheter stiffener adjustment system, comprising:
a first member having an outer diameter and having at least three first segments with at least one joint enabling the first segments to bend relative to each other;
a second member having an inner diameter defining a passageway through which the first member is movable relative to the second member, and having at least three second segments with at least one joint enabling the second segments to bend relative to each other;
in a first position, the first segments and the second segments are alignable substantially in phase to generate a first flexure condition; and
in a second position, the first segments and the second segments are alignable substantially out of phase to generate a second, stiffer flexure condition;
wherein the segments of the first member are disposed radially within the segments of the second member, and wherein translatable movement of the first member with respect to the second member in a longitudinal direction controls flexibility of the system, the joint for at least one of the first and second members is substantially helical.

2. (canceled)

3. (canceled)

4. The system of claim 1 wherein the at least one joint for at least one of the first and second members is formed by a thinning of material separating the segments while leaving remaining material between the segments.

5. The system of claim 1 wherein the at least one joint for at least one of the first and second members is formed by an elimination of material separating the segments along at least one side of one of the first and second members.

6. The system of claim 1 wherein at least one of the first and second members is substantially tubular.

7. The system of claim 1 wherein each of the first and second members is substantially cylindrical.

8. The system of claim 7 wherein the inner diameter of the second member is greater than the outer diameter of the first member in both the first and second positions.

9. A catheter stiffener adjustment system, comprising:
a first, substantially tubular member having an outer diameter and having at least three first segments with at least one joint enabling the first segments to bend relative to each other;
a second, substantially tubular member disposed substantially concentrically about the first member and having an inner diameter defining a passageway through which the first member is movable relative to the second member by at least one of rotation and translation, the inner diameter of the second member being greater than the outer diameter of the first member, and having at least three second segments with at least one joint enabling the second segments to bend relative to each other, at least the three first segments and the three second segments having sufficiently similar lengths to enable alignment substantially in phase with the first and second members in a first position and, in a second position, being alignable substantially out of phase;
in the first position, the first segments and the second segments are alignable substantially in phase to generate a first flexure condition; and
in the second position, the first segments and the second segments are alignable substantially out of phase to generate a second, stiffer flexure condition, the first member and the second member being at least one of rotatable and translatable relative to each other in both the first and second positions;
wherein the translatable movement of the first member with respect to the second member in a longitudinal direction controls flexibility of the system;
wherein the joint for each of the first and second members is substantially helical and substantially continuous.

10. (canceled)

11. A catheter having a stiffener adjustment system, comprising:
an outer catheter body having a central lumen;
a first member having an outer diameter and having at least three first segments with at least one joint enabling the first segments to bend relative to each other;
a second member movable through the central lumen of the catheter body and having an inner diameter defining a passageway through which the first member is movable relative to the second member, and having at least three second segments with at least one joint enabling the second segments to bend relative to each other;
in a first position, the first segments and the second segments are alignable substantially in phase to generate a first flexure condition for the catheter; and
in a second position, the first segments and the second segments are alignable substantially out of phase to generate a second, stiffer flexure condition for the catheter;
wherein translatable movement of the first member with respect to the second member in a longitudinal direction controls flexibility of the system;
wherein the joint for at least one of the first and second members is substantially helical and substantially continuous.

12. The catheter of claim 11 wherein the first member and the second member are at least one of rotatable and translatable relative to each other in both the first and second positions by moving an actuation mechanism connected to at least one of the first and second members.

13. (canceled)

14. (canceled)

15. A method of adjusting the stiffness of a catheter, comprising:
selecting a catheter having an outer catheter body defining a central lumen, the catheter further having a first member with an outer diameter and having at least three first segments with at least one joint enabling the segments to bend relative to each other, and a second member movable through the central lumen of the catheter body and having an inner diameter defining a passageway through which the first member is movable relative to the second member, and having at least three second segments with at least one joint enabling the segments to bend relative to each other, at least the three first segments and the three second segments having sufficiently similar lengths to enable alignment substantially in phase with the first and second members in a first position, in a second position, being alignable substantially out of phase;
aligning the first segments and the second segments substantially in phase to generate a first flexure condition for the catheter; and
aligning the first segments and the second segments substantially out of phase to generate a second, stiffer flexure condition for the catheter.

16. The method of claim 15 wherein aligning the first and second segments includes rotating at least one of the first member and the second member relative to each other.

17. The method of claim 15 wherein aligning the first and second segments includes translating at least one of the first member and the second member relative to each other.

18. The method of claim 15 wherein the joint for at least one of the first and second members is substantially helical and substantially continuous.

19. The method of claim 18 wherein at least one of the first and second members is substantially tubular.

20. The method of claim 15 wherein the catheter body has a plurality of longitudinal sections, with segments underlying at least one section to be capable of controlling flexibility for that section.
